(19) United States
(12) Patent Application Publication
(10) Pub. No.: US 2023/0405362 A1
(43) Pub. Date: Dec. 21, 2023

(54) SYSTEMS AND METHODS FOR TARGETED NEUROREGENERATION

(71) Applicant: THE TRUSTEES OF COLUMBIA UNIVERSITY IN THE CITY OF NEW YORK, New York, NY (US)

(72) Inventors: Elisa KONOFAGOU, New York, NY (US); Antonios POULIOPOULOS, London (GB)

(73) Assignee: THE TRUSTEES OF COLUMBIA UNIVERSITY IN THE CITY OF NEW YORK, New York, NY (US)

(21) Appl. No.: 18/459,591

(22) Filed: Sep. 1, 2023

Related U.S. Application Data
(63) Continuation of application No. PCT/US2022/018464, filed on Mar. 2, 2022.
(60) Provisional application No. 63/155,607, filed on Mar. 2, 2021.

(51) Int. Cl. A61N 7/00

(52) U.S. Cl. CPC A61N 7/00; A61N 2007/0026

(57) ABSTRACT

The present subject matter relates to techniques for treating a neurodegenerative disease. The disclosed system can include a transducer for stimulating a target tissue with focused ultrasound (FUS) and at least one nanocup. The transducer induces the FUS with a predetermined parameter to open the target tissue. The nanocup can include at least one gas pocket within a cavity of the nanocup and an effective amount of an active agent for neuroregeneration.

### CROSS-REFERENCE TO RELATED APPLICATION

This application is a continuation of International Patent Application No. PCT/US2022/018464, which was filed on Mar. 2, 2022, which claims priority to U.S. Provisional Patent Application No. 63/155,607, which was filed on Mar. 2, 2021, the entire contents of which are incorporated by reference herein.

### BACKGROUND

Neurodegenerative diseases such as Alzheimer's, Parkinson's, and amyotrophic lateral sclerosis can be characterized by a progressive loss of neuronal density and functionality. In Alzheimer's disease (AD), the underlying cause can be the gradual accumulation of amyloid-beta (A3) plaques and neurofibrillary tangles of hyperphosphorylated tau protein in the vicinity of neuronal bodies and axons. Amyloid plaques can be assessed using certain imaging techniques (e.g., amyloid PET imaging), which can suffer from poor resolution and involve radioactive tracers emitting ionizing electromagnetic radiation.

Certain therapies can be used for delaying the symptoms by reducing the rate of A3 and tau deposition. However, such therapy does not necessarily reverse the cognitive decline or reinstate the affected neural network. Furthermore, certain drugs having a large molecular weight, including antibodies or viral vectors, can be prevented from reaching their target for treating neurodegenerative diseases due to the presence of the blood-brain barrier (BBB).

Therefore, there is a need for improved techniques for treating neurodegenerative diseases.

### SUMMARY

The disclosed subject matter provides techniques for treating a neurodegenerative disease.

An example system can include a transducer for stimulating a target tissue with focused ultrasound (FUS) and at least one nanocup. In non-limiting embodiments, the transducer can be configured to induce the FUS with a predetermined parameter to open the target tissue. In certain embodiments, the nanocup can include at least one gas pocket within a cavity of the nanocup and an effective amount of an active agent for neuroregeneration.

In certain embodiments, the nanocup can be configured to react to the FUS with the predetermined parameter. In non-limiting embodiments, the nanocup can be configured to release the active agent after the FUS with the predetermined parameter is applied to the nanocup. In some embodiments, the size of the nanocup is less than about 100 nm.

In certain embodiments, the nanocup can include a PEG layer that is configured to conjugate with a protein and/or an antibody. In non-limiting embodiments, the nanocup comprises can include an anti-Aβ antibody. In some embodiments, the nanocup can have an Aβ binding efficiency more than about 80%.

In certain embodiments, the system can further include microbubbles configured to open the target tissue through cavitation. In non-limiting embodiments, the microbubbles can include at least one gas-filled cavity configured to act as a contrast agent in pulse inversion or full-waveform inversion ultrasound imaging.

In certain embodiments, the active agent can include a brain-derived neurotrophic factor (BDNF). In non-limiting embodiments, the BDNF can be covalently conjugated with the nanocup.

In certain embodiments, the system can include a processor configured to map a spatial distribution of a cavitation activity of the nanocup through passive acoustic mapping.

In certain embodiments, the predetermined parameter to open the target tissue can include a center frequency, an outer diameter, an inner diameter, a radius of curvature, or a combination thereof. In some embodiments, the center frequency can range from about 0.2 MHz to about 0.35 MHz.

In certain embodiments, the system can include a navigation guidance device configured to locate and/or monitor the target tissue. The navigation guidance device can include a cavitation detector configured to detect the microbubble cavitation and/or the nanocup cavitation.

The disclosed subject matter also provides methods for treating a neurodegenerative disease. An example method can include administering at least one nanocup to a target tissue and applying FUS using a transducer. The nanocup can include at least one gas pocket within a cavity of the nanocup and an effective amount of an active agent for neuroregeneration. In non-limiting embodiments, the transducer can be configured to induce the FUS with a predetermined parameter to open the target tissue. The predetermined parameter can include a center frequency, an outer diameter, an inner diameter, a radius of curvature, or a combination thereof.

In certain embodiments, the method can include imaging spatial distribution of the at least one nanocup through a full-waveform inversion imaging at various time points.

In certain embodiments, the method can include administering at least one microbubble and applying a low-pressure FUS to increase the permeability of the target tissue. The low-pressure can range from about 0.1 MPa to about 1 MPa.

In certain embodiments, the applying FUS includes applying a high-pressure FUS to trigger a release of an active agent from the nanocup. In non-limiting embodiments, the high-pressure can range from about 0.5 MPa to about 2 MPa.

In certain embodiments, the target tissue can include a cortical brain structure, a subcortical brain structure, a hippocampus, a caudate-putamen, a brain parenchyma, or a combination thereof.

The disclosed subject matter will be further described below.

### BRIEF DESCRIPTION OF THE DRAWINGS

FIG. 1 provides an image showing an example system for treating neurodegenerative diseases in accordance with the disclosed subject matter.

FIG. 2 provides a diagram showing an example targeted delivery of the nanocup for treating neurodegenerative diseases in accordance with the disclosed subject matter.

FIG. 3 provides an image showing an example nanocup for treating neurodegenerative diseases in accordance with the disclosed subject matter.

FIG. 4 provides a flow diagram showing an example method for treating neurodegenerative diseases in accordance with the disclosed subject matter.

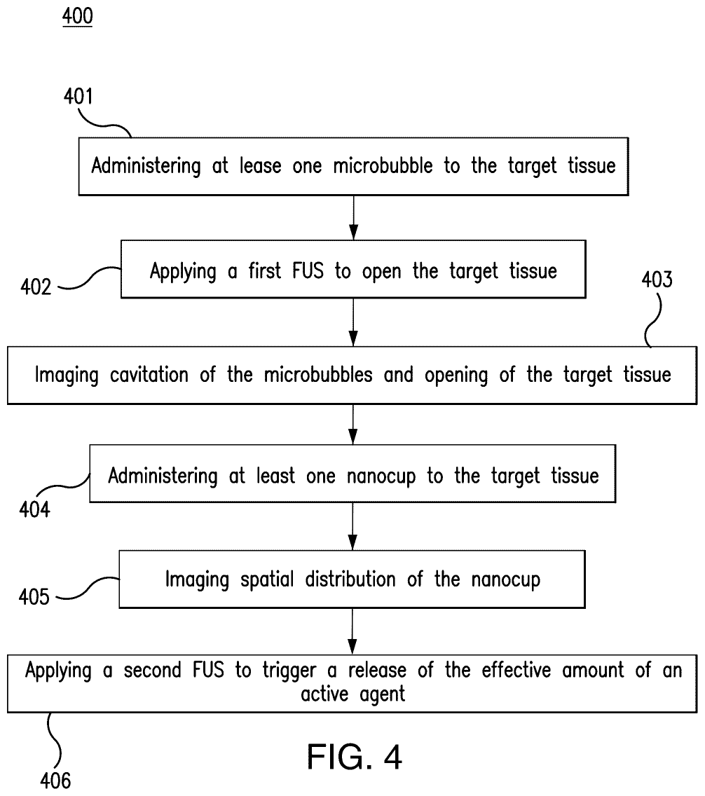

It is to be understood that both the foregoing general description and the following detailed description are exemplary and are intended to provide further explanation of the disclosed subject matter.

### DETAILED DESCRIPTION

The disclosed subject matter provides multilateral techniques for the diagnosis and treatment of neurodegeneration diseases. The disclosed subject matter provides systems and methods for treating neurodegeneration diseases through focused ultrasound (FUS)-mediated targeted delivery of multifunctional nanocups.

Unless otherwise defined, all technical and scientific terms used herein have the same meaning as commonly understood by one of ordinary skill in the art. In case of conflict, the present document, including definitions, will control.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within and within 2-fold, of a value.

The term “coupled,” as used herein, refers to the connection of a device component to another device component by methods known in the art.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like refer to obtaining a desired pharmacologic and/or physiologic effect. The effect can be prophylactic in terms of completely or partially preventing a disease or condition or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease or disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

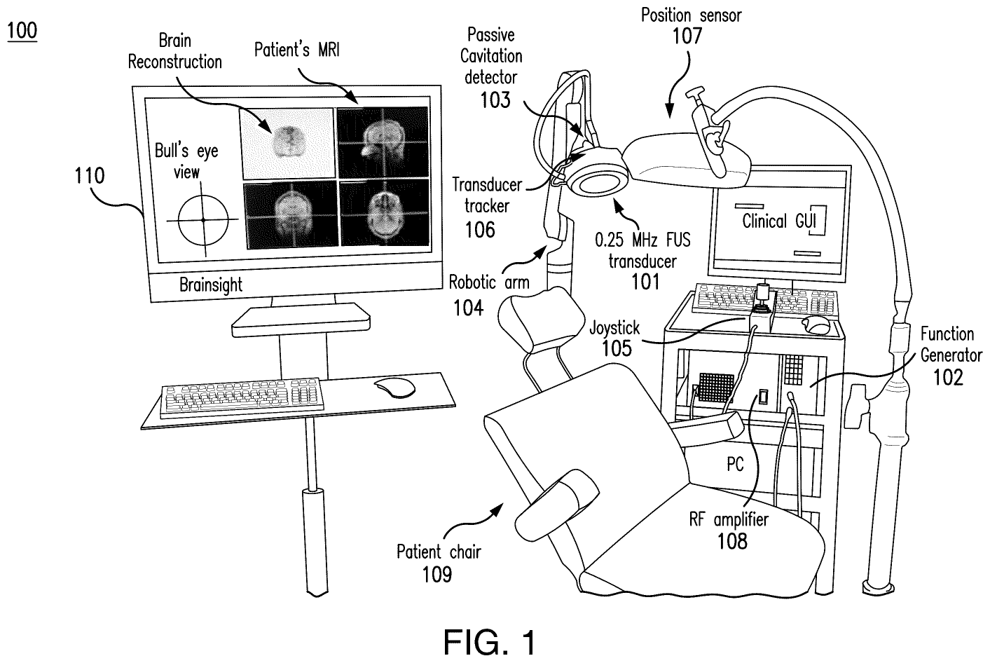

In certain embodiments, the disclosed subject matter provides a system for treating neurodegeneration diseases. An example system 100 can include a transducer for stimulating the target tissue with focused ultrasound (FUS) to open the target tissue and at least one nanocup. In non-limiting embodiments, as shown in FIG. 1, the transducer 101 can be a single element transducer 101 that can be configured to induce FUS for opening target tissue. For example, the single element transducer can generate an acoustic radiation force and induce cavitation at the target tissue. In some embodiments, the transducer can be connected to a function generator 102 and have a predetermined ultrasound parameter to induce cavitation, open the target tissue, and/or activate the nanocup. In non-limiting embodiments, the parameters can be modified or adjusted depending on a target tissue, a subject, a type of microbubbles, and/or a type of nanocups.

In certain embodiments, the predetermined ultrasound parameter can include a center frequency, a pressure, an outer diameter, an inner diameter, a radius of curvature, and a combination thereof. For example, the center frequency can range from about 20 kilohertz (kHz) to about 1 megahertz (MHz). In non-limiting embodiments, the center frequency can range from about 0.1 MHz to about 1 MHz, from about 0.1 MHz to about MHz, from about 0.1 MHz to about 0.35 MHz, from about 0.2 MHz to about 0.35 MHz, or from about 0.2 MHz to about 0.25 MHz. In non-limiting embodiments, the center frequency of the FUS stimulation probe can be about 0.2, 0.25, or 0.35 MHz. In non-limiting embodiments, the pulse length can range from about 5 μs to about 100 ms. In some embodiments, the pulse repetition frequency can range from about 1 Hz to about 5 kHz.

In certain embodiments, the predetermined ultrasound parameter can include outer diameter, inner diameter, and radius curvature of the disclosed element transducer. The outer diameter of the single element transducer can range from about 30 millimeters (mm) to about 200 mm, from about 30 mm to about 150 mm, from about 30 mm to about 110 mm, from about 40 mm to about 110 mm, from about 50 mm to about 110 mm, or from about 60 mm to about 110 mm. In non-limiting embodiments, the outer diameter of the single element transducer can be about 60 or 110 mm. In some embodiments, the inner diameter of the single element transducer can range from about 10 (mm) to about 60 mm, from about 10 mm to about 50 mm, from about 20 mm to about 50 mm, or from about 30 mm to about 50. In non-limiting embodiments, the inner diameter of the single element transducer can be about 44 mm. In some embodiments, the radius of curvature can range from about 30 millimeters (mm) to about 200 mm, from about 30 mm to about 150 mm, from about 30 mm to about 110 mm, from about 40 mm to about 110 mm, from about 50 mm to about 110 mm, from about 60 mm to about 110 mm, or from about 70 mm to about 110 mm. In non-limiting embodiments, the radius curvature can be about 70, 76, or 110 mm.

In certain embodiments, the predetermined ultrasound parameter can include a mechanical index, pulse length, pulse repetition frequency, peak-negative pressure, and sonication duration. The mechanical index can range from about 0.1 to about 1.9, from about 0.1 to about 1.5, from about 0.1 to about 1.0, from about 0.1 to about 0.9, from about 0.1 to about 0.8, from about 0.1 to about 0.7, from about 0.2 to about 0.7, from about 0.3 to about 0.7, or from about 0.4 to about 0.7. In non-limiting embodiments, the mechanical index can be about 0.4 or 0.8. The pulse length can range from about 0.001 milliseconds (ms) to about 100 ms, from about 0.001 ms to about 90 ms, from 0.001 ms to about 80 ms, from 0.001 ms to about 70 ms, from 0.001 ms to about 60 ms, from 0.001 ms to about 50 ms, from 0.001 ms to about 40 ms, from 0.001 ms to about 30 ms, from 0.001 ms to about ms, or from 0.001 ms to about 10 ms. In non-limiting embodiments, the pulse length can be about 10 ms. The pulse length can also range from about 1 cycle to about 5000 cycles, from about 1 cycle to about 4000 cycles, from about 1 cycle to about 10,000 cycles, from about 1 cycle to about 5000 cycles, from about 1 cycle to about 4000 cycles, from about 1 cycle to about 3000 cycles, from about 1 cycle to about 2500 cycles, from about 500 cycles to about 2500 cycles, from about 1000 cycles to about 2500 cycles, from about 1500 cycles to about 2500 cycles, or from about 2000 cycles to about 2500 cycles. In non-limiting embodiments, the pulse length can be about 2500 cycles. The pulse repetition frequency can range from about 0.1 Hz to about 10 kHz, from about 0.1 Hz to about 9 kHz, from about 0.1 Hz to about 8 kHz, from about 0.1 Hz to about 7 kHz, from about 0.1 Hz to about 6 kHz, from about 0.1 Hz to about 5 kHz, from about 0.1 Hz to about 4 kHz, from about 0.1 Hz to about 3 kHz, or from about 0.1 Hz to about 2 kHz. In non-limiting embodiments, the pulse repetition frequency can be about 2 Hz.

In certain embodiments, the sonication duration can range from about 0.1 minutes to about 5 minutes, from about 0.1 minutes to about 4 minutes, from about 0.1 minutes to about 3 minutes, from about 0.1 minutes to about 2 minutes, from about 0.5 minutes to about 2 minutes, or from about 1 minute to about 2 minutes. In non-limiting embodiments, the sonication duration can be about 2 minutes.

In certain embodiments, the peak-negative pressure can range from about 0.1 MPa to about 10 MPa, from about 0.1 MPa to about 9 MPa, from about 0.1 MPa to about 8 MPa, from about 0.1 MPa to about 7 MPa, from about 0.1 MPa to about 6 MPa, from about 0.1 MPa to about 5 MPa, from about 0.1 MPa to about 4 MPa, from about 0.1 MPa to about 3 MPa, from about 0.1 MPa to about 2 MPa, from about 0.1 MPa to about 1 MPa, from about 0.1 MPa to about 0.5 MPa, from about 0.1 MPa to about 0.4 MPa, from about 0.1 MPa to about 0.3 MPa, or from about 0.1 MPa to about 0.2 MPa. In non-limiting embodiments, the peak-negative pressure can be about 0.2 MPa.

In certain embodiments, the disclosed system can include microbubbles. The microbubbles can be configured to react to a predetermined pulse of the FUS and induce cavitation for opening the target tissue. The size of the microbubbles can range from about 1 micron to about 10 microns, from about 1 micron to about 9 microns, from about 1 micron to about 8 microns, from about 1 micron to about 7 microns, from about 1 micron to about 6 microns, from about 1 micron to about 6 microns, from about 1 micron to about 5 microns, from about 2 microns to about 5 microns, from about 3 microns to about 5 microns, or from about 4 microns to about 5 microns. In non-limiting embodiments, the dose of the microbubbles can be adjusted depending on the subject. For example, clinical doses (e.g., about 10111/kg) of the microbubbles for ultrasound imaging applications can be administered to a human subject.

In certain embodiments, the disclosed system can induce the cavitation of microbubbles to open the target tissue by applying a low-pressure FUS. For example, a low-pressure FUS (e.g., about 0.1 MPa to about 1 MPa) can induce the cavitation of microbubbles to open the blood-brain barrier of a subject.

In certain embodiments, the microbubbles are configured to carry or be coated with an active agent. The microbubbles can be configured to carry an active agent (e.g., small molecule) and be acoustically activated. For example, the molecule-carrying microbubbles can carry or be coated with medicinal molecules and/or a contrast agent and/or a biomarker and/or a liposome. Medicinal molecules and/or contrast agents can also be separately positioned in proximity to the targeted region. For example, the active agent can include a monoclonal antibody, a neuronal growth factor, a chemotherapeutic agent, or a combination thereof. In some embodiments, the FUS-induced microbubble cavitation can open the target tissue without damaging the target tissue.

In certain embodiments, the disclosed microbubbles can include at least one gas-filled cavity. The gas-filled cavity can act as a contrast agent in pulse inversion or full-waveform inversion ultrasound imaging. For example, the microbubble can expand and contract in response to the ultrasound imaging wave. The volumetric oscillations can produce acoustic signals, which can be mapped and localized in space. Using the gas-filled cavity, the disclosed system can be configured to image the spatial distribution of the microbubbles through in pulse inversion or full-waveform inversion imaging. For example, the disclosed system can include a processor that can map the spatial distribution of a cavitation activity of microbubbles through in pulse inversion or full-waveform inversion imaging. For example, the microbubbles can be imaged with pulse inversion as they are non-linear scatterers of ultrasound. The disclosed microbubbles can act as contrast agents in the brain ultrasound image and appear as hyperechoic regions. Their spatial distribution can be measured with certain beamforming techniques, such as delay and sum.

In certain embodiments, the target tissue can be any tissue. For example, the target tissue can be a nerve, a brain, a heart, muscle, tendons, ligaments, skin, vessels, blood-brain barrier, a subcortical brain structure, a hippocampus, a caudate-putamen, a brain parenchyma, or a combination thereof. In non-limiting embodiments, the target tissue can be a cortical and/or a subcortical region of a brain.

In certain embodiments, the subject can include a patient with neurodegenerative diseases (e.g., Alzheimer's, Parkinson's, and amyotrophic lateral sclerosis). In non-limiting embodiments, the disclosed subject matter can be applied to a patients with other brain indications (e.g., brain tumors and stroke) or other organs that can be targeted with molecular imaging.

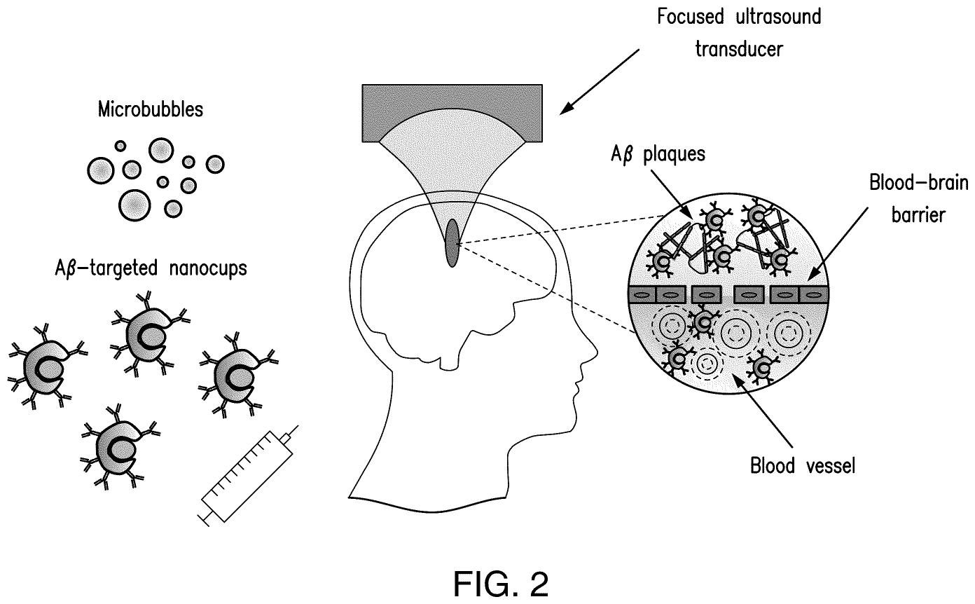

In certain embodiments, the disclosed system can include at least one nanocup. As shown in FIG. 2, the nanocup can be delivered to the target tissue through focused ultrasound (FUS)-mediated targeted delivery. For example, the permeability of the blood-brain barrier of the target tissue can increase by the FUS-mediated cavitation of microbubbles. The nanocup can be configured to pass through the blood-brain barrier with increased permeability.

In certain embodiments, the disclosed nanocups can be coated with antibodies targeting the amyloid plaques, tau neurofibrillary tangles, or brain tumors. FUS-induced BBB opening can allow delivery of the nanocups into the brain parenchyma. Because of their molecular targeting, nanocups can preferentially accumulate on the amyloid plaques, in the case of Alzheimer's disease.

In certain embodiments, the nanocup can be a polymeric nanocup with entrapped gas pockets within its cavity. In non-limiting embodiments, the nanocup can be produced through a seeded polymerization technique using polystyrene (PS) as the core material and methyl methacrylate (MMA) as coating material. Following a drying phase and resuspension, the template nanocup can be deformed, forming an air cavity within the crevice.

In certain embodiments, the nanocups can have a diameter from about 1 nm to about 100 nm, from about 1 nm to about 90 nm, from about 1 nm to about 80 nm, from about 1 nm to about 70 nm, from about 1 nm to about 60 nm, from about 1 nm to about 50 nm, from about 1 nm to about 40 nm, from about 1 nm to about 30 nm, from about 1 nm to about 20 nm, from about 1 nm to about 10 nm, from about 20 nm to about 100 nm, from about 20 nm to about 100 nm, from about 30 nm to about 100 nm, from about 40 nm to about 100 nm, from about 50 nm to about 100 nm, from about 60 nm to about 100 nm, from about 70 nm to about 100 nm, from about 80 nm to about 100 nm, or from about 90 nm to about 100 nm.

In non-limiting embodiments, the nanocup can include at least one gas pocket within a cavity of the nanocup. The gas-filled cavity of the nanocup can be configured to act as a contrast agent in pulse inversion or full-waveform inversion ultrasound imaging. The nanobubble can expand and contract in response to the ultrasound imaging wave. The volumetric oscillations can produce acoustic signals, which can be mapped and localized in space. The cavity can provide ultrasound contrast and cavitation response under therapeutic ultrasound exposure. Using the gas-filled cavity, the disclosed system can be configured to image the spatial distribution of the nanocup through in pulse inversion or full-waveform inversion imaging. For example, the disclosed system can include a processor that can map the spatial distribution of a cavitation activity of nanocup through in pulse inversion or full-waveform inversion imaging. The nanobubbles can be imaged with pulse inversion as they are non-linear scatterers of ultrasound. Nanobubbles can act as contrast agents in the brain ultrasound image and appear as hyperechoic regions. Their spatial distribution can be measured with certain beamforming techniques, such as delay and sum. The contrast of the disclosed nanocups can be assessed through a phantom using a B-mode and a pulse inversion ultrasound imaging. In certain embodiments, the disclosed system can include a processor that can map the spatial distribution of cavitation activity and nanocups using passive acoustic mapping. Passive acoustic mapping can be used to locate the activity of cavitation agents during FUS treatment. The disclosed nanocups exposed to therapeutic ultrasound can produce their own acoustic emissions, which can be passively captured by an imaging array and then processed with passive acoustic mapping algorithms. The outcome can be a passive acoustic map of nanocup activity, which can be correlated with the spatial distribution of the induced bioeffect.

In some embodiments, the nanocup can include an effective amount of an active agent for neuroregeneration. For example, the nanocup can be coated with brain-derived neurotrophic factor (BDNF), which can be released after ultrasound-triggered activation of the nanocup. In non-limiting embodiments, the active agent (e.g., BDNF) can be covalently conjugated with the nanocups via EDC chemistry. The conjugation of the active agent and size distribution of the nanobubbles can be assessed through electron transmission microscopy, FTIR spectroscopy, X-ray absorption fine structure analysis, or confocal microscopy.

In certain embodiments, the disclosed system can induce the release of the active agent from the nanobubble by applying a high-pressure FUS. For example, a high-pressure FUS (e.g., about 0.5 MPa to about 2 MPa) can induce the release of the active agent from the nanocups. At high acoustic pressures, the nanocup oscillations can become increasingly non-stable and larger in amplitude. The induced stresses within the nanocup can release the active agent above a stress threshold.

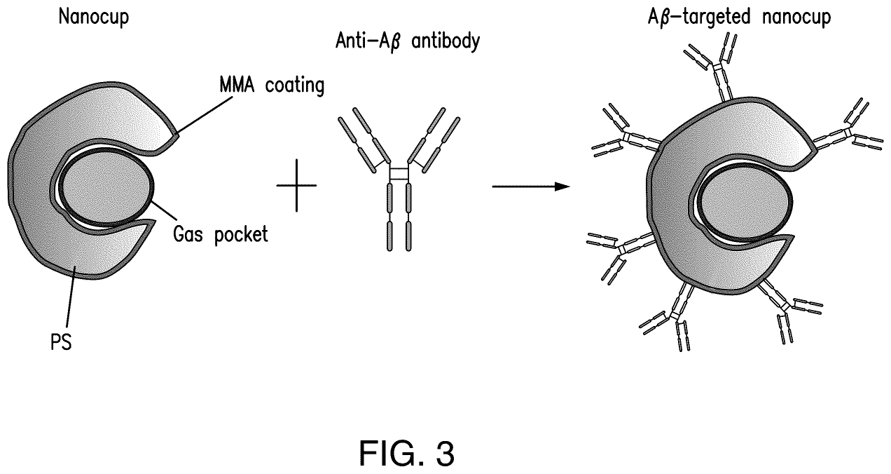

In certain embodiments, as shown in FIG. 3, the nanocup can include a protein and/or an antibody. In non-limiting embodiments, the nanocup can be coated with a protein and/or an antibody. In some embodiments, the nanocup can be conjugated with a protein and/or an antibody. For example, the nanocup can be coated with amyloid-beta (Aβ) that has an affinity for the Aβ (4-10) epitope. In non-limiting embodiments, a PEG layer can be added in order to facilitate antibody and protein conjugation.

In certain embodiments, the antibody can be screened. For example, genetically humanized VelocImmune (VI) mice can be used for screening potent anti-Aβ antibodies binding onto the Aβ(4-10) epitope. The mice can be immunized with DNA plasmids expressing Aβ3. Titers of blood can be collected from the mice, and the resulting antibodies can be screened for binding affinity to the Aβ(4-10) epitope through ELISA. In non-limiting embodiments, the antibody with the desired binding affinity can be selected and labeled with a fluorescent marker (e.g., FITC). In certain embodiments, the selected antibody can be conjugated with the polymeric nanocups. The nanocups with the anti-Aβ antibodies can provide the Aβ binding efficiency more than about 80%.

In certain embodiments, the disclosed system can include a navigation guidance device that can be configured to locate and/or monitor the target tissue. The navigation guidance device can include a cavitation detector 103 and an arm 104. In non-limiting embodiments, the navigation guidance device can be an image-based navigator device.

In certain embodiments, the cavitation detector 103 can be configured to detect the FUS-induced cavitation in real-time. In non-limiting embodiments, the cavitation detector can be a passive cavitation detector (PCD) co-aligned with the single element transducer. The PCD can have certain imaging parameters that can allow the detection of cavitation signals through a bone (e.g., human skull). For example, the imaging parameter can include a center frequency, a diameter, and a focal depth. The center frequency of the PCD can range from about 0.1 megahertz (MHz) to about 10 MHz, from about 0.1 MHz to about 9 MHz, from about 0.1 MHz to about 8 MHz, from about 0.1 MHz to about 7 MHz, from about 0.1 MHz to about 6 MHz, from about 0.1 MHz to about 5 MHz, from about 0.1 MHz to about 4 MHz, from about 0.1 MHz to about 3 MHz, or from about 0.1 MHz to about 2 MHz. In non-limiting embodiments, the center frequency of the PCD can be about 1.5 MHz. The diameter of the PCD can range from about 10 millimeters (mm) to about 60 mm, from about 10 mm to about 50 mm, from about 10 mm to about 40 mm, from about 20 mm to about 40 mm, or from about 30 mm to about 40 mm. In non-limiting embodiments, the diameter of the PCD can be about 32 mm. The focal depth of the PCD can range from about 30 millimeters (mm) to about 200 mm, from about 30 mm to about 150 mm, from about 40 mm to about 150 mm, from about 50 mm to about 150 mm, or from about 100 mm to about 150 mm. In non-limiting embodiments, the focal depth of the PCD can be about 114 mm.

In certain embodiments, the PCD can detect the cavitation signals to determine the types/modes of the cavitation. For example, the PCD can detect harmonic peaks, ultra harmonic peaks, broadband emissions, cavitation magnitude, cavitation duration, and a microbubble velocity to identify stable or inertial cavitation. In stable cavitation, the microbubble expands and contracts with the acoustic pressure rarefaction and compression over several cycles, and such action can result in the displacement of the vessel diameter through dilation and contraction. In inertial cavitation, the bubble can expand to several factors greater than its equilibrium radius and subsequently collapse due to the inertia of the surrounding media, thus also inducing a potential alteration of the vascular physiology. The PCD can detect the cavitation signals that can be used for calculating stable harmonic, stable ultra harmonic, and inertial cavitation levels.

In certain embodiments, the navigation guidance device includes an arm 104. In non-limiting embodiments, the single element transducer 101 co-aligned with the cavitation detector 103 can be attached to arm 104. The arm can be a robotic arm with 4 degrees of freedom. The movement of the robotic arm can be controlled by a controller 105 (e.g., a joystick).

In certain embodiments, the image-based navigator device can be configured to image the target tissue and reconstruct a 3D image before and after the application of the FUS. The 3D skin scalp and brain reconstructions can allow the accurate placing of the focal volume in the targeted region. The planned and achieved trajectory can be visualized in real-time.

In certain embodiments, the disclosed system can further include a transducer tracker 106, a position sensor 107, a radiofrequency amplifier 108, a portable chair 109, and a display 110. The transducer and subject trackers can include infrared light-reflecting spheres and be configured to perform real-time monitoring of the transducer's and subject's position in space. The radiofrequency can amplify an amplification (e.g., 55-dB) of the signal generated by the function generator before application onto the single-element transducer.

In certain embodiments, the disclosed system can include a processor coupled to the single element transducer and/or the navigation guidance device. In non-limiting embodiments, the processor can be coupled to the probes directly (e.g., wire connection or installation into the probes) or indirectly (e.g., wireless connection). The processor can be configured to perform the instructions specified by software stored in a hard drive, a removable storage medium, or any other storage media. The software can include computer codes, which can be written in a variety of languages, e.g., MATLAB and/or Microsoft Visual C++. Additionally or alternatively, the processor can include hardware logic, such as logic implemented in an application-specific integrated circuit (ASIC). The processor can be configured to control one or more of the system components described above. For example, and as embodied herein, the processor can be configured to control imaging and ultrasound stimulation. The processor can be configured to control the output of the function generator and/or the transducer to provide the FUS to the subject. Additionally, or alternatively, the processor can be configured to map the cavitation activities of nanocups and microbubbles.

In certain embodiments, the processor can be configured to analyze the detected cavitation signals and determine a mode of the cavitation. The processor can analyze cavitation signals that are measured by the cavitation detector. For example, the processor can calculate stable harmonic, stable ultra harmonic, and inertial cavitation levels by analyzing harmonic peaks, ultra harmonic peaks, broadband emissions, a cavitation magnitude, a cavitation duration, and microbubble velocity signals detected by the PCD. Cavitation doses can be calculated as the sum of cavitation levels throughout the treatment duration. Stable cavitation doses can quantify the magnitude of stable and recurrent cavitation, while inertial cavitation doses can quantify the magnitude of transient inertial cavitation. The relative weighting of stable vs. inertial cavitation can be a safety determinant for ultrasound treatments.

In certain embodiments, the disclosed subject matter provides a method for treating a neurodegenerative disease. An example method 400 can include administering at least one nanocup to a target tissue 404 and applying FUS using a transducer to the target tissue 406. The nanocup comprises at least one gas pocket within a cavity of the nanocup. The nanocup comprises an effective amount of the disclosed active agent for neuroregeneration. In non-limiting embodiments, the predetermined parameter can include the disclosed center frequency, outer diameter, inner diameter, radius of curvature, or a combination thereof.

In certain embodiments, the disclosed method can further include imaging spatial distribution of the at least one nanocup through a full-waveform inversion imaging at various time points 405. For example, the nanocup can include a gas-filled cavity that can be configured to act as a contrast agent in pulse inversion or full-waveform inversion ultrasound imaging. The cavity can provide ultrasound contrast and cavitation response under therapeutic ultrasound exposure. Using the gas-filled cavity, the spatial distribution of the nanocup can be mapped through in pulse inversion or full-waveform inversion imaging. In non-limiting embodiments, the target tissue and the distribution of nanocups/microbubble can be imaged following the FUS treatment at different time points.

In certain embodiments, the disclosed method can further include administering at least one microbubble 401 and applying a low-pressure FUS to increase the permeability of the target tissue 402. In non-limiting embodiments, a low-pressure FUS (e.g., from about 0.1 MPa to about 1 MPa) can induce the cavitation of microbubbles to open the blood-brain barrier of a subject. The application of the low-pressure FUS and microbubble can be a temporary treatment and non-invasively increase the permeability of the target tissue. In non-limiting embodiments, the method can include imaging the cavitation of the microbubbles and opening of the target tissue 403.

In certain embodiments, the disclosed method can further include applying a high-pressure FUS to trigger a release of an active agent from the nanocup 406. In non-limiting embodiments, a high-pressure FUS (e.g., about 0.5 MPa to about 2 MPa) can induce the release of the disclosed active agent from the disclosed nanocups.

Due to the size, it can be challenging to deliver a particle/vehicle with a drug to the target tissue. The disclosed subject matter can provide improved targeted delivery techniques by using FUS, microbubbles, and/or nanocups. The disclosed nanocups can be imaged and activated through the human skull and in centimeter depths. Using the disclosed nanocups with antibodies, amyloid plaques can be imaged (e.g., at micrometer resolution) through various imaging techniques (e.g., amyloid PET). The disclosed ultrasound-triggered localized release can enhance the therapeutic action of the disclosed active agent (e.g., BDNF), which can have a short circulation time if administered without a vehicle. With the disclosed techniques, the delivered dose can increase at the vicinity of amyloid plaques and, thus, at the site of intense neuronal depletion.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A system for treating a neurodegenerative disease, comprising:
a transducer for stimulating a target tissue with focused ultrasound (FUS), wherein the transducer induces the FUS with a predetermined parameter to open the target tissue; and
at least one nanocup, wherein the nanocup comprises at least one gas pocket within a cavity of the nanocup, wherein the nanocup comprises an effective amount of an active agent for neuroregeneration.

2. The system of claim 1, wherein the nanocup is configured to react to the FUS with the predetermined parameter.

3. The system of claim 1, wherein a size of the nanocup is less than about 100 nm.

4. The system of claim 1, further comprising microbubbles configured to open the target tissue through cavitation.

5. The system of claim 5, wherein the microbubbles comprise at least one gas-filled cavity configured to act as a contrast agent in pulse inversion or full-waveform inversion ultrasound imaging.

6. The system of claim 1, wherein the active agent comprises a brain-derived neurotrophic factor (BDNF).

7. The system of claim 6, wherein the BDNF is covalently conjugated with the nanocup.

8. The system of claim 1, wherein the nanocup is configured to release the active agent after the FUS with the predetermined parameter is applied to the nanocup.

9. The system of claim 1, further comprising a processor configured to map a spatial distribution of a cavitation activity of the nanocup through passive acoustic mapping.

10. The system of claim 1, wherein the nanocup comprises a PEG layer that is configured to conjugate with a protein and/or an antibody.

11. The system of claim 1, wherein the nanocup comprises an anti-Aβ antibody.

12. The system of claim 11, wherein the nanocup has an Aβ binding efficiency more than about 80%.

13. The system of claim 1, wherein the predetermined parameter to open the target tissue is selected from the group consisting of a center frequency, an outer diameter, an inner diameter, a radius of curvature, and a combination thereof.

14. The system of claim 12, wherein the center frequency ranges from about 0.2 MHz to about 0.35 MHZ.

15. The system of claim 1, further comprising a navigation guidance device configured to locate and/or monitor the target tissue, wherein the navigation guidance device comprises a cavitation detector configured to detect the microbubble cavitation and/or the nanocup cavitation.

16. A method for treating a neurodegenerative disease, comprising:
administering at least one nanocup to a target tissue, wherein the nanocup comprises at least one gas pocket within a cavity of the nanocup, wherein the nanocup comprises an effective amount of an active agent for neuroregeneration; and
applying FUS using a transducer, wherein the transducer induces the FUS with a predetermined parameter to open the target tissue, the predetermined parameter is selected from the group consisting of a center frequency, an outer diameter, an inner diameter, a radius of curvature, and a combination thereof.

17. The method of claim 16, further comprising imaging spatial distribution of the at least one nanocup through a full-waveform inversion imaging at various time points.

18. The method of claim 16, further comprising administering at least one microbubble, and applying a low-pressure FUS to increase a permeability of the target tissue, wherein the low-pressure ranges from about about 0.1 MPa to about 1 MPa.

19. The method of 18, wherein the applying FUS comprises applying a high-pressure FUS to trigger a release of an active agent from the nanocup, wherein the high-pressure ranges from about about 0.5 MPa to about 2 MPa.

20. The method of claim 18, wherein the target tissue comprises a cortical brain structure, a subcortical brain structure, a hippocampus, a caudate putamen, a brain parenchyma, or a combination thereof.
